(19) United States
(12) Patent Application Publication
(10) Pub. No.: US 2024/0172927 A1
(43) Pub. Date: May 30, 2024

(54) BENDING PORTION OF ENDOSCOPE, ENDOSCOPE, AND MANUFACTURING METHOD OF BENDING PORTION OF ENDOSCOPE

(71) Applicant: OLYMPUS MEDICAL SYSTEMS CORP., Tokyo (JP)

(72) Inventors: Shohei UEHARA, Tokyo (JP); Masaya ABE, Sagamihara-shi (JP); Shingo HORII, Sagamihara-shi (JP); Kei MATSUOKA, Tokyo (JP); Yoshihiro YOKOMAE, Tokyo (JP)

(73) Assignee: OLYMPUS MEDICAL SYSTEMS CORP., Tokyo (JP)

(21) Appl. No.: 18/520,620

(22) Filed: Nov. 28, 2023

Related U.S. Application Data
(60) Provisional application No. 63/428,172, filed on Nov. 28, 2022.

(51) Int. Cl. A61B 1/008; A61B 1/00; A61B 1/005

(52) U.S. Cl. CPC A61B 1/008; A61B 1/0011; A61B 1/0055; A61B 1/0057

(57) ABSTRACT

A bending portion of an endoscope according to the disclosure includes: a plurality of bending pieces rotatably coupled to each other by a coupling fastener; a wire configured to transmit a traction operating force to a first bending piece included in the plurality of bending pieces; and a pin located in a first hole in the first bending piece, the pin connecting the wire to the first bending piece, a first end portion of the pin protruding from an outer circumference of the first bending piece, a second end portion of the pin protruding from an inner circumference of the first bending piece, a third portion of the pin contacts a surface of the first hole. A first width dimension of the first end portion is larger than an opening dimension of the first hole, the wire is attached to the second end portion.

### RELATED APPLICATION DATA

This application is based on and claims priority under 35 U.S.C. § 119 to U.S. Provisional Application No. 63/428,172 filed on Nov. 28, 2022, the entire contents of which are incorporated herein by reference.

### FIELD OF THE DISCLOSURE

The present disclosure relates to a bending portion of an endoscope, an endoscope, and a manufacturing method of a bending portion of an endoscope and, in particular, the present disclosure relates to a fixing structure of a bending wire in a bending portion of an endoscope.

### DESCRIPTION OF RELATED ART

Conventionally, endoscopes have been widely used in the medical field, the industrial field, and the like. A medical endoscope used in the medical field includes a function of inserting an insertion portion equipped with an image pickup unit into a body cavity of a living body and acquiring an image of a lesion or the like inside an organ or the like. An image acquired in this manner is used for observation of the lesion, image diagnosis by an examination, and the like.

An insertion portion of a conventional endoscope of this type is constructed by coupling a distal end portion, a bending portion, and a flexible tube in this order from a distal end side. In addition, the insertion portion has flexibility as a whole and is formed in an elongated tubular shape. Among these portions, the bending portion is configured so as to be actively bendable in accordance with an operation by a user. Therefore, for example, the bending portion is constructed so as to include a plurality of bending pieces, a bending wire, and the like.

Each of the plurality of bending pieces is formed in an approximately annular shape and the annular bending pieces are coupled in a direction along a long axis of the insertion portion to form a bending tube with an approximately tubular shape. An inside of the bending portion (bending tube) allows insertion of the bending wire.

One end of the bending wire is fixed to the bending piece on a distalmost side. Another end of the bending wire is fixed to an operating member on an operation portion side. According to such a configuration, when the user operates the operating member, a traction force of the bending wire is transmitted to the bending portion. Accordingly, the bending portion bends in a predetermined direction in accordance with the operation by the operating member. A configuration of a bending portion in a conventional endoscope is generally a configuration such as described above.

In addition, in a conventional endoscope, as fixing means of the one end of the bending wire and the bending piece on the distalmost side, for example, fixing is generally performed using brazing or the like. For example, Japanese Patent No. 4436503, Japanese Patent Application Laid-Open Publication No. H7-134253, and the like disclose a fixing structure configured to fix one end of a bending wire and a bending piece on a distalmost side to each other using brazing or the like.

### SUMMARY OF THE DISCLOSURE

A bending portion for use with an endoscope according to an aspect of the present disclosure includes: a plurality of bending pieces, adjacent bending pieces of the plurality of bending pieces are rotatably coupled to each other by at least one coupling fastener; a wire configured to transmit a traction operating force to a first bending piece included in the plurality of bending pieces; and a pin located in a first hole in the first bending piece, a first end portion of the pin protrudes from the first hole past an outer circumference surface of the first bending piece, a second end portion of the pin protrudes from the first hole past an inner circumference surface of the first bending piece, a third portion of the pin is between the first end portion and the second end portion, the third portion of the pin contacts a surface of the first hole. The wire is attached to the second end portion.

A manufacturing method of a bending portion of an endoscope according to an aspect of the present disclosure includes: coupling a first bending piece and a second bending piece to each other with a coupling fastener, the first bending piece including a first hole extending through a circumferential wall of the first bending piece; fixing a first end portion of a pin to a first end of a wire, the pin having a length which exceeds a thickness of the circumferential wall of the first bending piece; inserting the pin into the first hole from an inner side of the first bending piece to position a second end portion of the pin to protrude from the first hole past an outer circumference surface of the circumferential wall of the first bending piece; and attaching the pin to the first bending piece.

### BRIEF DESCRIPTION OF THE DRAWINGS

FIG. 1 is a schematic view showing an overall configuration of an endoscope according to an embodiment of the present disclosure.

FIG. 2 is an external perspective view showing a configuration of a bending portion of the endoscope according to the embodiment of the present disclosure.

FIG. 3 is a sectional view taken along a virtual plane indicated by arrow reference signs [3] in FIG. 2.

FIG. 4 is an external perspective view showing, in an isolated manner, only a bending piece on a distalmost side in the bending portion shown in FIG. 2.

FIG. 5 is an external perspective view showing a wire locking pin (single component prior to mounting) in the bending pieces shown in FIG. 2.

FIG. 6 is an external perspective view showing a state where one end of a bending wire is locked by the wire locking pin shown in FIG. 5.

FIG. 7 is an enlarged sectional view of a substantial part showing, in an enlarged manner, a part of FIG. 3 (a section in a vicinity of a distal end side of the bending portion).

FIG. 8 is an explanatory diagram of a manufacturing method of the bending portion of the endoscope according to the embodiment of the present disclosure (an enlarged sectional view of a substantial part in a state where a plurality of bending pieces are coupled by a coupling shaft).

FIG. 9 is an explanatory diagram of the manufacturing method of the bending portion of the endoscope according to the embodiment of the present disclosure (a diagram showing a situation where one end of a bending wire is inserted through a through-hole of a wire locking pin).

FIG. 10 is an explanatory diagram of the manufacturing method of the bending portion of the endoscope according to the embodiment of the present disclosure (a diagram showing a situation where the first portion A and the second portion B of the wire locking pin are inserted through a lateral hole of the first bending piece).

FIG. 11 is an explanatory diagram of the manufacturing method of the bending portion of the endoscope according to the embodiment of the present disclosure (a diagram showing a situation prior to pressing and fastening the first portion A of the wire locking pin).

FIG. 12 is an explanatory diagram of the manufacturing method of the bending portion of the endoscope according to the embodiment of the present disclosure (a diagram showing a situation after the first portion A of the wire locking pin has been pressed and fastened).

FIG. 13 is an external perspective view showing a wire locking pin (single component prior to mounting) in a bending piece of an endoscope of another embodiment of the present disclosure.

FIG. 14 is an external perspective view showing a state where one end of a bending wire is locked by the wire locking pin shown in FIG. 13.

FIG. 15 is a view showing a situation where a through-hole is deformed by applying swaging to the wire locking pin shown in FIG. 14.

FIG. 16 is an enlarged sectional view of a substantial part showing, in an enlarged manner, a section in a vicinity of a distal end side of a bending portion, in the other embodiment of the present disclosure.

### DETAILED DESCRIPTION

Generally, in a configuration in which brazing or the like is used as a fixing structure of a bending wire in a bending portion of a conventional endoscope, holding portions (for example, a groove, a hole, or a concave portion) for holding the bending wire prior to fixing with respect to a bending piece on a distalmost side must be formed in advance.

In addition, for example, a brazing operation is performed in a state where one end of the bending wire is being held by the holding portions. Since the brazing operation performed in this case is a time- and labor-consuming precision operation, there is a problem in that the brazing operation is not suitable for mass production and the brazing operation causes an increase in a burden of manufacturing cost.

Furthermore, in recent years, so-called single-use endoscopes that are disposed after a single use have become popular. A single-use endoscope of this type requires a thorough reduction in manufacturing cost.

Meanwhile, there is a tendency in recent endoscopes toward further narrowing of diameters. Therefore, since an assembly operation of a narrow-diameter endoscope requires operations of higher precision, such an assembly operation is a cause for higher cost.

As described above, in a conventional endoscope and, in particular, in a single-use endoscope, there is a desire to avoid adopting a fixing structure or the like accompanying, for example, a brazing operation as much as possible in order to realize a reduction in manufacturing cost.

According to an embodiment of the present disclosure to be described below, a bending portion of an endoscope, an endoscope, and a manufacturing method of a bending portion of an endoscope can be provided which realize a simple structure an assembly operation of which is not time- and labor-consuming in a fixing structure of a bending wire in the bending portion of the endoscope to enable a contribution to be made toward reducing manufacturing cost and to realize a structure that can be readily mass-produced.

Hereinafter, the present disclosure will be described using the illustrated embodiment.

Each drawing used in the following description is a schematic representation and, in order to show each constituent element in a size large enough to recognize the constituent element on the drawings, a dimensional relationship, a scale, or the like of each member may be shown differentiated for each constituent element. Therefore, in terms of a quantity of each constituent element, a shape of each constituent element, a ratio of sizes among respective constituent elements, a relative positional relationship among respective constituent elements, and the like described in each drawing, the present disclosure is not limited to only illustrated modes.

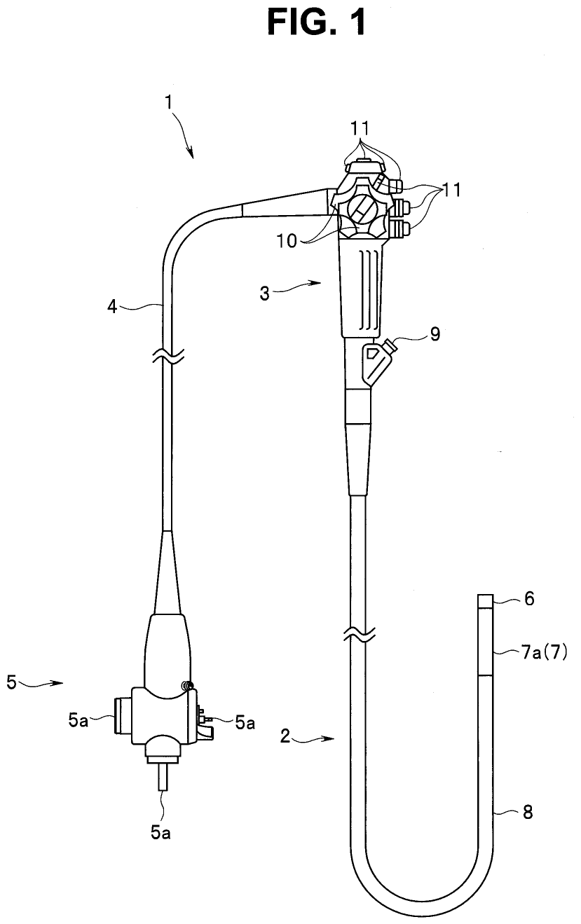

First, a schematic configuration of an endoscope according to an embodiment of the present disclosure will be described below with reference to FIG. 1. FIG. 1 is a schematic view showing an overall configuration of the endoscope according to the embodiment of the present disclosure.

An endoscope 1 shown in FIG. 1 is an endoscope capable of acquiring image data of a desired observation site such as a lesion inside a subject and represents an example of a configuration of an endoscope an object of examination, observation, and treatment of which is mainly an inside of a gastrointestinal system.

The endoscope 1 is mainly constituted of an insertion portion 2, an operation portion 3, a universal cord 4, an endoscope connector 5, and the like.

The insertion portion 2 is a constituent member to be inserted into a lumen of the subject. The insertion portion 2 has flexibility and is formed in an elongated tubular shape. The insertion portion 2 is constituted of a distal end portion 6, a bending portion 7, and a flexible tube portion 8. The distal end portion 6, the bending portion 7, and the flexible tube portion 8 are sequentially coupled from a distal end side of the insertion portion 2.

The distal end portion 6 is arranged at a distal end of the insertion portion 2. The distal end portion 6 is constituted of a base member which internally includes a predetermined space and which is formed of a rigid member, various constituent units respectively arranged at predetermined positions in the internal space of the base member, and the like. For example, a plurality of constituent units (not illustrated) such as an image pickup unit, an illuminating unit, and a gas feeding/liquid feeding unit are arranged inside the distal end portion 6. Individual configurations of the constituent units are portions not directly related to the present disclosure. Therefore, a description and illustration of detailed configurations of the constituent units will be omitted. It is assumed that constituent units of a same type as constituent units applied to a conventional endoscope of a same type are to be applied as the constituent units.

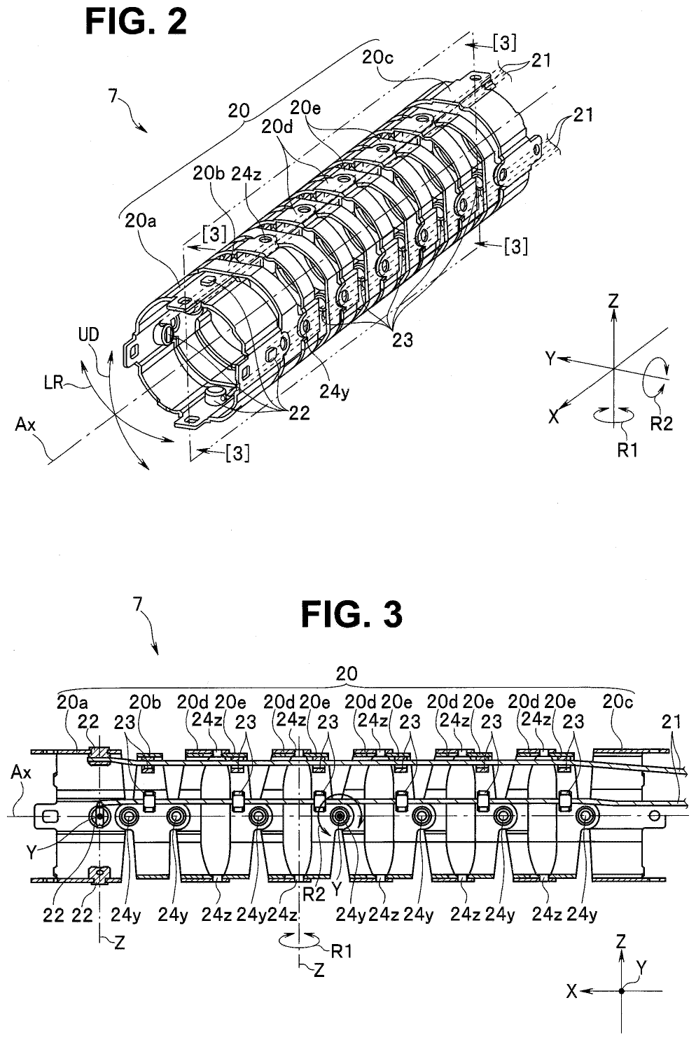

The bending portion 7 is a constituent unit having a structure that enables the bending portion 7 to be actively bendable in accordance with a predetermined operation by a user of the endoscope 1. A cover member 7a that is a tubular elastic body is mounted to an outer circumference of the bending portion 7. A detailed configuration of the bending portion 7 will be described later (refer to FIG. 2 and subsequent drawings).

The flexible tube portion 8 is a long tubular constituent member which has flexibility. Cables and the like (for example, an image pickup signal line, an image pickup control signal line, an optical fiber cable, and an illumination signal line; all not illustrated) that are extended from the respective constituent units (for example, the image pickup unit and the illuminating unit; all not illustrated) that are provided in the distal end portion 6, a gas feeding/liquid feeding conduit of the gas feeding/liquid feeding unit, a treatment instrument insertion channel (not illustrated), and the like are inserted inside the flexible tube portion 8.

The operation portion 3 is a constituent unit that is arranged at a proximal end side of the insertion portion 2. In addition to a treatment instrument insertion opening 9, a bending operating member 10, and the like, the operation portion 3 is provided with a plurality of operating members 11 for performing various types of operations. Since individual functions and configurations of the plurality of operating members 11 are also portions not directly related to the present disclosure, detailed descriptions will be omitted.

The treatment instrument insertion opening 9 is an opening into which a treatment instrument (not illustrated) is to be inserted. The treatment instrument insertion opening 9 is communicated with a treatment instrument insertion channel (not illustrated) that is inserted through inside of the insertion portion 2.

The bending operating member 10 is an operating member for operating bending of the bending portion 7. In the illustrated example, the bending operating member 10 represents an example of a configuration in which a rotating operating member is provided in plurality. More specifically, the endoscope 1 shown in FIG. 1 represents an example in which, for example, the bending portion 7 is constituted of two operating members, namely, an operating member that causes the bending portion 7 to bend in a left-right direction and an operating member that causes the bending portion 7 to bend in an up-down direction.

Note that, in this case, the left-right direction and the up-down direction refer to a left-right direction (screen horizontal direction) and an up-down direction (screen vertical direction) in a screen frame of an image obtained by the endoscope 1. As will be described later, the bending portion 7 that is a bending tube is bendable around a long axis Ax (refer to a direction of an arrow LR, a direction of an arrow UD, and the like in FIG. 2). In this case, when the bending portion 7 bends in the direction LR shown in FIG. 2, an image movement in the left-right direction is observed as an endoscopic image. Therefore, the bending direction in this case is to be referred to as the left-right direction. In a similar manner, for example, when the bending portion 7 bends in the direction UD shown in FIG. 2, an image movement in the up-down direction is observed as an endoscopic image. Therefore, the bending direction in this case is to be referred to as the up-down direction.

The universal cord 4 is a cord that extends from the operation portion 3. The endoscope connector 5 is provided at a distal end of the universal cord 4. An image pickup signal line or the like (not illustrated) extended from the image pickup unit, an image pickup control signal line (not illustrated) that connects the image pickup unit to a control unit (not illustrated) or a video processor (not illustrated), an optical fiber cable that provides connection from a light source apparatus (not illustrated) to the illuminating unit, a gas feeding/liquid feeding conduit (not illustrated), and the like are inserted inside the universal cord 4.

The endoscope connector 5 is consecutively provided at a distal end of the universal cord 4. The endoscope connector 5 includes a plurality of connectors 5a such as a light source connector, a video connector, and a liquid feeding tube. A detailed description of each connector 5a will be omitted on the assumption that connectors of a similar type as connectors applied to a conventional endoscope of a same type are to be applied as the connectors. This concludes the description of a general configuration of the endoscope 1.

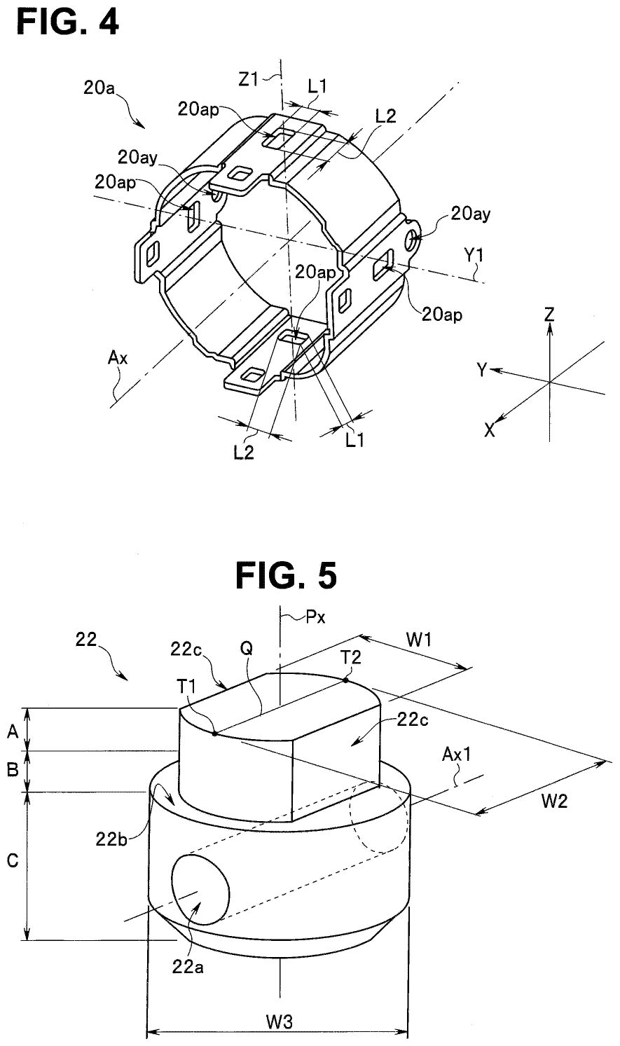

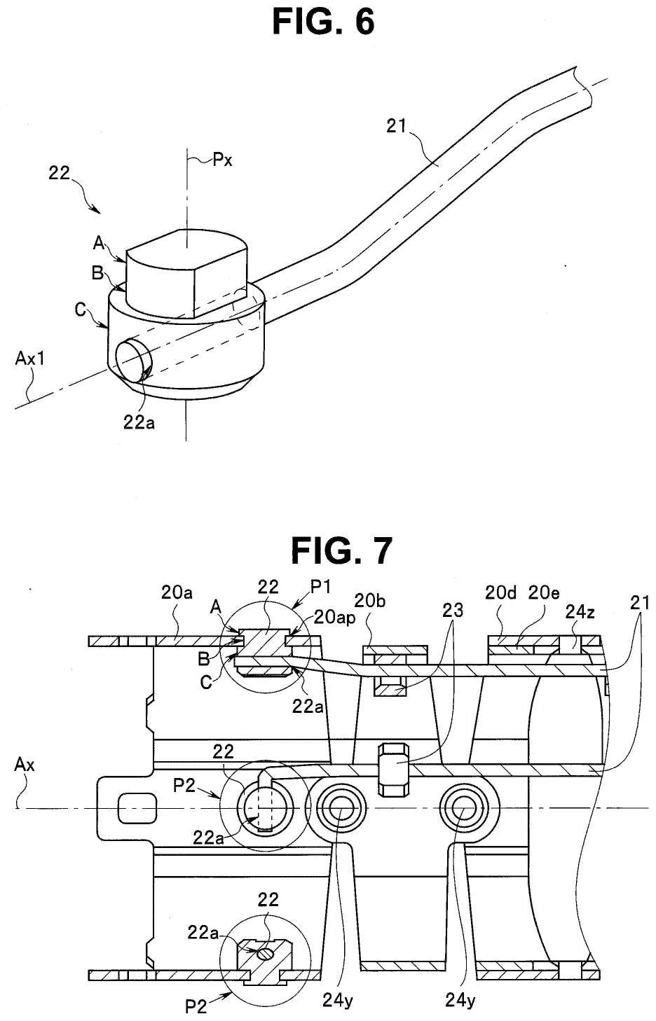

Next, a configuration of the bending portion of the endoscope according to the embodiment of the present disclosure will be described below with reference to FIGS. 2 to 7. FIGS. 2 to 7 are diagrams showing a configuration of the bending portion of the endoscope according to the embodiment of the present disclosure. FIG. 2 is an external perspective view showing, in an isolated manner, the bending portion of the endoscope according to the embodiment of the present disclosure. FIG. 3 is a sectional view showing a cross section taken along a virtual plane indicated by arrow reference signs [3] in FIG. 2. FIG. 4 is an external perspective view showing, in an isolated manner, only a bending piece on a distalmost side in the bending portion shown in FIG. 2. FIG. 5 is an external perspective view showing a wire locking pin in a state (single component) prior to being mounted to the bending piece. FIG. 6 is an external perspective view showing a state where one end of a bending wire is locked by the wire locking pin shown in FIG. 5. FIG. 7 is an enlarged sectional view of a substantial part showing, in an enlarged manner, a part of FIG. 3 (a section in a vicinity of a distal end side of the bending portion).

The bending portion 7 is constituted of a plurality of bending pieces 20, a plurality of bending wires 21, a plurality of wire locking pins 22, a plurality of wire guide members 23, and the like.

As shown in FIGS. 2 and 3, the plurality of bending pieces 20 include a first bending piece (referred to as a specific bending piece) 20a, a second bending piece 20b, a third bending piece 20c, a fourth bending piece 20d, and a fifth bending piece 20e.

Each of the plurality of bending pieces 20 (20a, 20b, 20c, 20d, and 20e) is formed in an approximately annular shape. In this case, for example, the plurality of bending pieces 20 are integrally formed from a metal plate with a predetermined thickness. Known techniques are used as manufacturing means for forming the plurality of bending pieces. Therefore, a detailed description of the manufacturing means will be omitted.

Each of the plurality of bending pieces 20 (20a, 20b, 20c, 20d, and 20e) is coupled by a predetermined construction in a direction along a long axis of the insertion portion 2 (a direction in which the insertion portion 2 is inserted into the subject; hereinafter, referred to as a long axis direction). Accordingly, the bending portion 7 constitutes a bending tube with an approximately tubular shape as a whole.

In this case, a reference sign Ax shown in FIGS. 2, 3, and the like denotes a long axis of the bending portion 7 forming the bending tube. The long axis Ax of the bending portion 7 approximately matches the long axis of the insertion portion 2. Note that among coordinate axes indicated by reference signs X, Y, and Z in FIGS. 2 and 3, the long axis Ax of the bending portion 7 is indicated by the reference sign X. In addition, two axes that are orthogonal to the long axis Ax and orthogonal to each other are denoted by the reference signs Y and Z.

The first bending piece 20a is a bending piece arranged on a distalmost side of the bending portion 7. The second bending piece 20b is a bending piece that is adjacent on a proximal end side of the first bending piece 20a and that is coupled to the first bending piece 20a. The third bending piece 20c is a bending piece arranged on a proximalmost side of the bending portion 7. The fourth bending piece 20d and the fifth bending piece 20e are bending pieces that are arranged side by side in plurality between the second bending piece 20b and the third bending piece 20c. The plurality of bending pieces 20 is arranged in a longitudinal direction of the bending portion 7 to form a longitudinally arranged plurality of bending pieces. The first bending piece 20a is located at a distalmost end of the longitudinally arranged plurality of bending pieces 20. The first bending piece 20a includes a second hole that opposes a first hole across a longitudinal axis of the longitudinally arranged plurality of bending pieces.

Note that the fourth bending piece 20d and the fifth bending piece 20e are arranged adjacent to and coupled to each other. In other words, the fourth bending piece 20d and the fifth bending piece 20e are alternately arranged between the second bending piece 20b and the third bending piece 20c.

In this case, a proximal end side of the fourth bending piece 20d and a distal end side of the fifth bending piece 20e are turnably coupled within a predetermined range in a predetermined direction (a direction indicated by an arrow R1 in FIG. 3) around a coupling shaft (coupling fastener or coupling body) that is approximately parallel to the Z axis. The coupling fastener may include screw, rivet, etc. The coupling fastener has a coupling surface.

In a similar manner, a distal end side of the fourth bending piece 20d and a proximal end side of the fifth bending piece 20e are turnably coupled within a predetermined range in a predetermined direction (a direction indicated by an arrow R2 in FIG. 3) around a coupling shaft (coupling fastener) that is approximately parallel to the Y axis.

Bending pieces that are adjacent to each other are coupled using two rivets 24y and two rivets 24z. Of the two rivets 24y (coupling fastener), one each is provided at each of positions that oppose each other across the long axis Ax on an axis approximately parallel to the Y axis. In a similar manner, of the two rivets 24z (coupling fastener), one each is provided at each of positions that oppose each other across the long axis Ax on an axis approximately parallel to the Z axis.

Note that in FIG. 2, illustration of the rivets themselves are omitted and only holes through which the rivets are to be inserted are illustrated in order to prevent the drawing from becoming complicated. In addition, reference signs 24y and 24z have been attached to the holes in FIG. 2. In this case, axial directions of the coupling shafts approximately match long axes of the respective rivets 24y and 24z.

The first bending piece 20a and the second bending piece 20b are coupled by the rivets 24y that are a coupling fastener (first coupling fastener). To this end, holes 20ay (refer to FIG. 4) to allow insertion of the rivets 24y are formed in the first bending piece 20a.

In addition, a bending piece on a distalmost side among the plurality of fourth bending pieces 20d is coupled by the rivets 24y that are a coupling shaft to the second bending piece 20b. Furthermore, a bending piece on a proximalmost side among the plurality of fifth bending pieces 20e is coupled by the rivets 24y that are a coupling shaft to the third bending piece 20c.

In this manner, among the plurality of bending pieces 20, adjacent bending pieces are rotatably coupled to each other using rivets 24y and rivets 24z that are coupling shafts and are configured to turn within a predetermined range of a turn R1 around the Z axis or a turn R2 around the Y axis. According to this configuration, the bending portion 7 that is a bending tube is configured as a whole to be bendable in the direction of the arrow LR or in the direction of the arrow UD in FIG. 2 around the long axis Ax.

A coupling structure of the plurality of bending pieces 20 such as the coupling structure described above is a known technique applied in conventional endoscopes.

The bending wire 21 for realizing a bending operation of the bending portion 7 is inserted inside the bending portion 7 constituted of a bending tube configured as described above. The bending wire 21 is a long member to be inserted inside the insertion portion 2 from the bending portion 7 to the operation portion 3. In the endoscope 1 according to the present embodiment, an example constructed by providing a total of four bending wires including two bending wires that contribute toward bending in the up-down direction and two bending wires that contribute toward bending in the left-right direction is shown. One end of the bending wire 21 is fixed to an inner side of the first bending piece 20a that is the bending piece on a distalmost side (the specific bending piece). In addition, another end of the bending wire 21 is fixed by predetermined means at a predetermined position (not illustrated) to the bending operating member 10 inside the operation portion 3. According to such a configuration, when the user operates the bending operating member 10, a traction operating force of the bending wire 21 is transmitted to the first bending piece 20a of the bending portion 7. Accordingly, the bending portion 7 bends in a predetermined direction. The bending wire 21 can be of any suitable material, so it includes metal, plastic, etc.

In each of the plurality of bending pieces 20, the wire guide member 23 is welded to and arranged at a predetermined section on an inner side of each bending piece 20. The wire guide member 23 has an approximately annular shape and has a through-hole to allow insertion of the bending wire 21. In other words, the wire guide member 23 is a member that functions as a guide portion for guiding reciprocal movement of the bending wire 21 in a predetermined direction (a direction approximately parallel to a direction along the long axis Ax).

As shown in FIGS. 2 and 3, the wire guide member 23 is arranged in plurality at predetermined intervals in a circumferential direction on an inner circumferential surface of the plurality of fifth bending pieces 20e. In the present embodiment, four wire guide members 23 are provided in accordance with the number of arranged bending wires 21.

More specifically, a total of four wire guide members 23 are provided on an inner side of each of the fifth bending pieces 20e at respective positions in each vicinity of the two rivets 24y and each vicinity of the two rivets 24z. In this case, the four wire guide members 23 are fixed at positions separated from each other by predetermined intervals (for example, angular intervals of approximately 90 degrees in the circumferential direction) in a circumferential direction around a direction in which the plurality of coupling shafts (the rivets 24y and the rivets 24z) are lined up (in other words, a direction along the long axis Ax). In addition, after the bending wire 21 is inserted through the through-hole of each wire guide member 23, one end of the bending wire 21 is held by the wire locking pin 22.

The wire locking pin 22 is a member configured to hold one end of the bending wire 21 and to fix the bending wire 21 to the first bending piece 20a by being fixed to the first bending piece 20a. To this end, the first bending piece 20a is provided with a plurality of lateral holes 20ap (refer to FIG. 4) for arranging and fixing the wire locking pin 22. In FIG. 4, the plurality of lateral holes 20ap has a lateral shape, but the first bending piece 20a can include other shape of a hole for arranging and fixing the wire locking pin 22. The hole may be round, non-round, or various polygons.

The plurality of lateral holes 20ap are formed in an approximately rectangular shape. In this case, the plurality of lateral holes 20ap include lateral holes 20ap a short side L1 of which (refer to FIG. 4) is arranged approximately parallel to a direction along the long axis Ax and lateral holes 20ap a long side L2 of which (refer to FIG. 4) is arranged approximately parallel to a direction along the long axis Ax. An arrangement of the short side L1 and the long side L2 of the lateral hole 20ap is appropriately set in accordance with an internal arrangement of the bending wire 21 or the like. Changing the arrangement of the short side L1 and the long side L2 of the lateral hole 20ap enables an arrangement direction of a through-hole 22a of the wire locking pin 22 (to be described in detail later) to be appropriately set.

The lateral hole 20ap is provided in the same number as the number of the provided bending wires 21. In the present embodiment, as shown in FIG. 4, an example in which four lateral holes 20ap are provided in the first bending piece 20a is shown.

In this case, as shown in FIG. 4, two of the lateral holes 20ap are provided at positions that oppose each other across the long axis Ax of the bending portion 7 on an axis (refer to reference sign Y1 in FIG. 4) that is approximately parallel to the Y axis in the first bending piece 20a. In addition, two of the lateral holes 20ap are also provided at positions that oppose each other across the long axis Ax of the bending portion 7 on an axis (refer to reference sign Z1 in FIG. 4) that is approximately parallel to the Z axis. In this case, the axis Y1 represents a straight line that connects respective centers of the two lateral holes 20ap. In a similar manner, the axis Z1 represents an axial line that is approximately orthogonal to the axis Y1 and that is a straight line that connects respective centers of the other two lateral holes 20ap.

In the present embodiment, an endoscope that accommodates bending in four directions including up-down directions and left-right directions is provided as described above. Accordingly, the bending portion 7 of the endoscope 1 according to the present embodiment is constructed by providing the four bending wires 21. In accordance with the four bending wires 21, four lateral holes 20ap of the first bending piece 20a are provided. In addition, the wire locking pin 22 (a total of the four wire locking pins 22) is arranged in each of the four lateral holes 20ap.

Note that the number of the arranged lateral holes 20ap is appropriately changed in accordance with the number of the bending wires 21. For example, in a case of an endoscope adopting a two-direction bending system that accommodates either bending in up-down directions or bending in left-right directions, two bending wires are provided. In this case, a configuration in which two lateral holes 20ap are provided on either the axis Y1 or the axis Z1 may be adopted. For this reason, at least two of the lateral holes 20ap of the first bending piece 20a need only be provided at opposing positions.

As shown in FIG. 5 and the like, the wire locking pin 22 is formed so as to include a first portion A (first end portion), a second portion B (third end portion), and a third portion C (second end portion). The wire locking pin 22 extends in a longitudinal direction from a first end surface of the first end portion to a second end surface of the second end portion. An axis of the through-hole 22a can intersect the longitudinal direction of the pin.

In this case, the first portion A is a portion that is arranged so as to protrude toward an outer circumferential side of the first bending piece 20a in a state where the wire locking pin 22 is mounted to the first bending piece 20a (refer to FIG. 7). In addition, the second portion B is a portion that is arranged at a position corresponding to the lateral hole 20ap of the first bending piece 20a in the same state shown in FIG. 7. The second portion B can contact a surface of the lateral hole 20ap. Furthermore, the third portion C is a portion that is arranged so as to protrude toward an inner circumferential side of the first bending piece 20a in the same state shown in FIG. 7.

Although details will be described later, the first portion A corresponds to a portion that is pressed and deforms when the wire locking pin 22 is mounted to the lateral hole 20ap of the first bending piece 20a. Therefore, in a state prior to the wire locking pin 22 being mounted to the lateral hole 20ap of the first bending piece 20a or, in other words, in a state where the wire locking pin 22 is present as a single component, the first portion A and the second portion B are integrally formed in a same shape and same dimensions as shown in FIGS. 5 and 6.

A mode of the wire locking pin 22 prior to being mounted to the first bending piece 20a is formed as follows. As shown in FIGS. 5 and 6, the first portion A and the second portion B of the wire locking pin 22 are integrally formed in a mode in which a part of an approximately columnar shape has been cut away.

More specifically, for example, as shown in FIG. 5, the first portion A and the second portion B are formed in a columnar shape having two planes 22c that oppose each other across a long axis Px of the wire locking pin 22 and an approximately elliptical cross section in which respective end portions of the two planes are connected by an arc with a predetermined curvature. In this case, a distance of separation between the two planes is assumed to be a first width W1. In addition, a distance of separation of two points T1 and T2 at which a straight line Q being approximately parallel to the two planes and passing through the long axis Px intersects with arc-shaped outer edges of the first portion A and the second portion B (a distance of separation between vertices of the respective arcs) is assumed to be a second width W2. At this point, the first width W1 is smaller than the second width W2 (second width W2>first width W1; refer to FIG. 5).

The third portion C is formed in an approximately columnar shape. In this case, a maximum width (second width dimension) W3 of the third portion C is formed larger than at least a width (opening dimension) of the long side L2 of the lateral hole 20ap (width W3>width of long side L2 of lateral hole). The second width dimension may be in a common orientation with the opening dimension.

A maximum width of the second portion B (the second width W2) is formed approximately equal to or slightly smaller than the width (maximum width) of the long side L2 of the lateral hole 20ap (width of long side L2 of lateral hole≥ second width W2). In a similar manner, a minimum width of the second portion B (the first width W1) is formed approximately equal to or slightly smaller than the width (minimum width) of the short side L1 of the lateral hole 20ap (width of short side L1 of lateral hole≥first width W1). Accordingly, the second portion B is insertable into the lateral hole 20ap.

In addition, a maximum width W3 of the third portion C is formed approximately equal to or slightly larger than the maximum width of the second portion B (the second width W2) (width W3≥second width W2). In other words, the third portion C forms an outward flange with respect to the first portion A and the second portion B.

The maximum width (the second width W2) and the minimum width (the first width W1) of the first portion A of the wire locking pin 22 as a component prior to mounting has a same shape and same dimensions as the second portion B. Therefore, shapes of the first portion A and the second portion B become shapes that can be inserted into the lateral hole 20ap when the first width W1 approximately matches the short side L1 of the lateral hole 20ap and the second width W2 approximately matches the long side L2 of the lateral hole 20ap.

In addition, when the first portion A and the second portion B of the wire locking pin 22 are inserted into the lateral hole 20ap of the first bending piece 20a, the insertion of the wire locking pin 22 is locked as a flange surface 22b of the third portion C abuts against an inner circumferential surface of the first bending piece 20a. Accordingly, when the wire locking pin 22 is mounted to the first bending piece 20a, the third portion C is arranged so as to protrude toward an inner circumferential side of the first bending piece 20a.

As will be described later, the first portion A of the wire locking pin 22 is pressed and deforms when the wire locking pin 22 is mounted to the lateral hole 20ap of the first bending piece 20a. In this manner, the width of the first portion A (first width dimension) when the first portion A deforms becomes larger than the width of the lateral hole 20ap (opening dimension) as shown in FIG. 7 (details will be described later).

In addition, the through-hole 22a is formed in the third portion C. The through-hole 22a is formed so as to penetrate the third portion C in a direction along an axis Ax1 that is orthogonal to the long axis Px of the wire locking pin 22. As shown in FIG. 6, the through-hole 22a is a portion configured to hold the bending wire 21 when one end of the bending wire 21 is inserted. At this point, the bending wire 21 is held between inner wall surfaces of the through-hole 22a. The bending wire 21 may be fixed to the wire locking pin 22 by any suitable means, such as by friction fit, crimping, adhesive, welding, soldering, fusing or a combination of them.

When the wire locking pin 22 is mounted to the first bending piece 20a, for example, the through-hole 22a of the wire locking pin 22 is to be arranged in different aspects such as an aspect indicated by a reference sign P1 in FIG. 7 and an aspect indicated by a reference sign P2 in FIG. 7.

In an arrangement aspect of the through-hole 22a of the wire locking pin 22 indicated by the reference sign P1 in FIG. 7, the through-hole 22a penetrates the third portion C in a direction approximately parallel to a direction along the long axis Ax of the bending portion 7. To this end, the lateral hole 20ap of the first bending piece 20a corresponding to the wire locking pin 22 to be mounted at this position is arranged so that the long side L2 is approximately parallel to a direction along the long axis Ax.

On the other hand, in an arrangement aspect of the through-hole 22a of the wire locking pin 22 indicated by the reference sign P2 in FIG. 7, the through-hole 22a penetrates the third portion C in a direction that is inclined with respect to a direction along the long axis Ax of the bending portion 7. The example shown in FIG. 7 is an example in which the through-hole 22a is arranged in a direction approximately orthogonal to the long axis Ax. When the through-hole 22a is arranged in a direction approximately orthogonal to the long axis Ax of the bending portion 7 as indicated by the reference sign P2, the bending wire 21 can be arranged so as to avoid the rivets 24y adjacent to the wire locking pin 22 (the rivets that connect the first bending piece 20a and the second bending piece 20b to each other). Arranging the through-hole 22a in this manner prevents the bending wire 21 from sliding on the rivets 24y and enables a traction force of the bending wire 21 to be reduced.

In this manner, the arrangement of the through-hole 22a of the wire locking pin 22 is appropriately set according to an arrangement mode of the lateral hole 20ap of the first bending piece 20a.

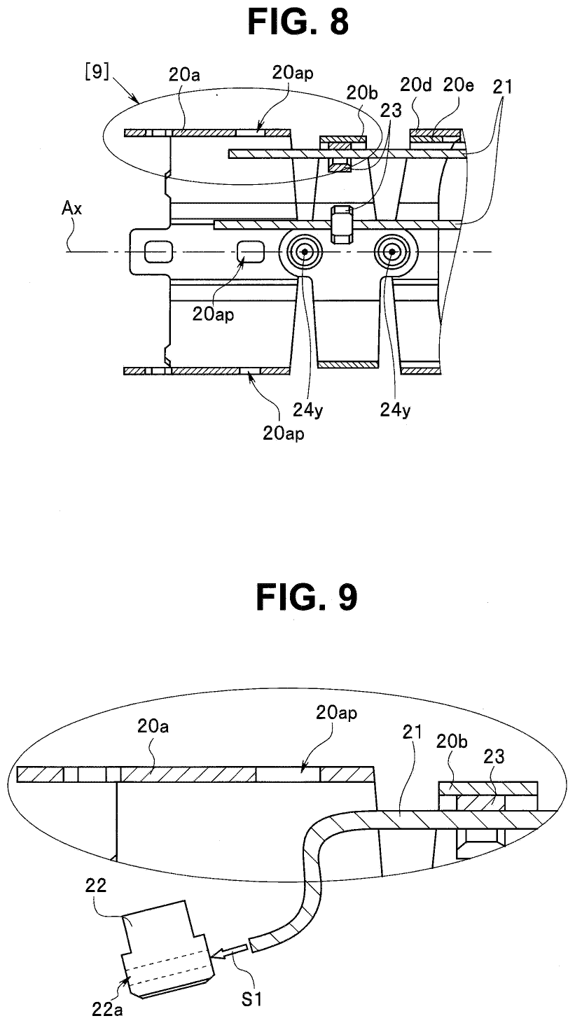

A manufacturing method of the bending portion of the endoscope according to the present embodiment configured in this manner will be briefly described below. FIGS. 8 to 12 are diagrams for describing the manufacturing method of the bending portion of the endoscope according to the embodiment of the present disclosure. Among the drawings, FIG. 8 shows a state where a plurality of bending pieces including the first bending piece and the second bending piece are coupled by coupling shafts. Note that FIG. 8 is an enlarged sectional view of a substantial part showing, in an enlarged manner, a region including a distal end-side bending piece of the bending portion shown in FIG. 3.

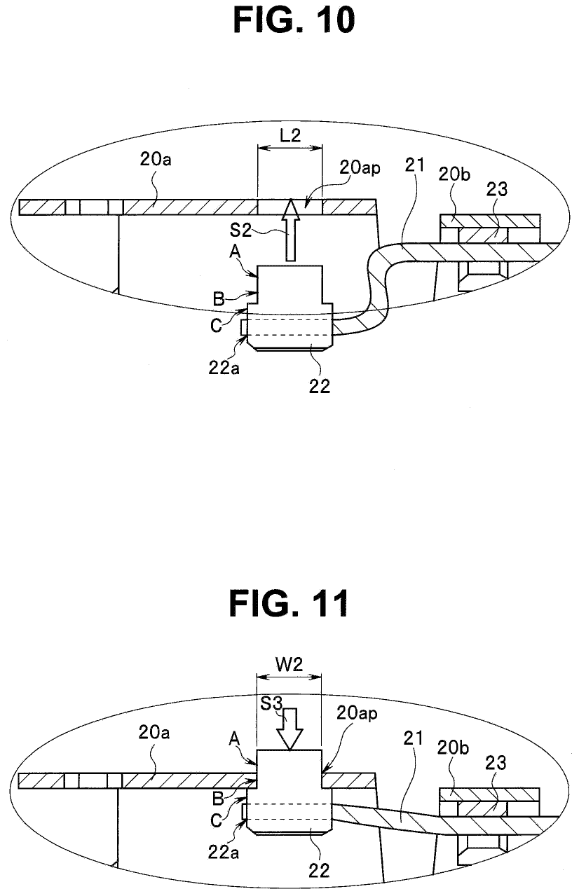

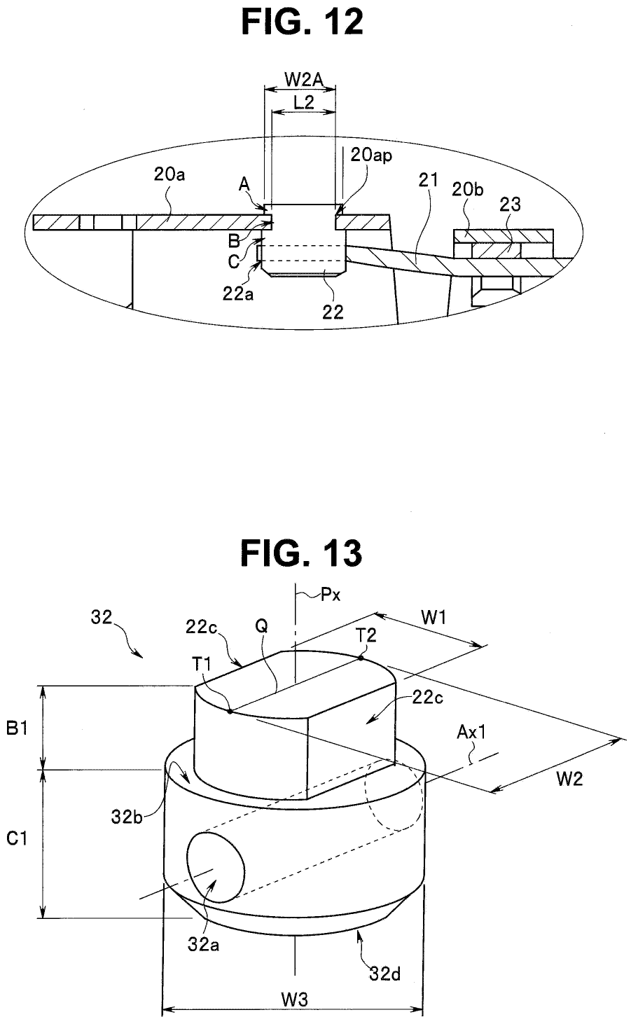

FIGS. 9 to 12 are enlarged sectional views of a substantial part showing, in an enlarged manner, a region denoted by a reference sign [9] in FIG. 8 and sequentially show procedures of the manufacturing method of the bending portion. FIG. 9 shows a situation after FIG. 8 where one end of the bending wire is inserted through the through-hole of the wire locking pin. FIG. 10 shows a situation after FIG. 9 where the first portion A and the second portion B of the wire locking pin are inserted through the lateral hole of the first bending piece. FIG. 11 shows a situation after FIG. 10 when the first portion A of the wire locking pin is pressed and fastened. FIG. 12 shows a situation after FIG. 11 after the first portion A of the wire locking pin has been pressed and fastened.

First, each of the plurality of bending pieces 20 that constitute the bending portion 7 is coupled. For example, the annular first bending piece 20a including the lateral hole 20ap and the annular second bending piece 20b are coupled by the coupling shaft (rivets 24y). Next, the second bending piece 20b and the fourth bending piece 20d are coupled by the coupling shaft (rivets 24y). In a similar manner, the fourth bending piece 20d and the fifth bending piece 20e are coupled by the coupling shaft (rivets 24z). In a similar manner, the fourth bending piece 20d and the fifth bending piece 20e are alternately coupled in plurality. Finally, the fifth bending piece 20e and the third bending piece 20c are coupled by the coupling shaft (rivets 24y) (refer to FIG. 3). In this manner, a bending tube made up of the plurality of bending pieces 20 is formed.

Inside the bending tube formed in this manner, each of the plurality of bending wires 21 is inserted through the wire guide member 23 at each predetermined position. In this case, the bending wire 21 is inserted from the distal end side to the proximal end side of the bending portion 7 (bending tube). Alternatively, a procedure may be adopted in which the bending wire 21 is inserted from the proximal end side to the distal end side of the bending portion 7 (bending tube). In this manner, the plurality of bending wires 21 are inserted through predetermined sections inside the bending tube constructed by coupling the plurality of bending pieces 20. A state at this point is the state shown in FIG. 8.

Next, the wire locking pin 22 is attached in a predetermined mode to one end of each of the plurality of bending wires 21 shown in FIG. 8. In other words, as shown in FIG. 9, one end of the bending wire 21 is inserted through the through-hole 22a of the wire locking pin 22 (refer to a direction of an arrow S1 in FIG. 9). In this manner, one end of the bending wire 21 is inserted through the through-hole 22a of the wire locking pin 22. A state where one end of the bending wire 21 is inserted through the through-hole 22a is as shown in FIGS. 6 and 10.

Next, the wire locking pin 22 in which one end of the bending wire 21 is inserted through the through-hole 22a of the wire locking pin 22 is inserted into the lateral hole 20ap from an inner circumferential side of the first bending piece 20a (refer to a direction of an arrow S2 in FIG. 10). Accordingly, as shown in FIG. 11, the wire locking pin 22 is arranged at a position where the first portion A protrudes toward an outer circumferential side from the lateral hole 20ap of the first bending piece 20a. In addition, the second portion B is arranged at a position corresponding to the lateral hole 20ap of the first bending piece 20a. Furthermore, the third portion C is arranged at a position that protrudes toward an inner circumferential side from the lateral hole 20ap of the first bending piece 20a. In this manner, the wire locking pin 22 assumes a state shown in FIG. 11.

While an assembly procedure of attaching the wire locking pin 22 to one end of the bending wire 21 after inserting the bending wire 21 through the wire guide member 23 will be disclosed in the present embodiment, an assembly procedure is not limited to the assembly procedure. For example, the wire locking pin 22 may be attached to one end of the bending wire 21 first and another end of the bending wire 21 may be inserted through the wire guide member 23 of the second bending piece 20b and the wire guide member 23 on the proximal end side. Accordingly, even when the other end of the bending wire 21 is erroneously pulled by a force that is stronger than necessary, the wire locking pin 22 is snagged by the distal end portion of the bending portion 7 and the bending wire 21 is prevented from becoming detached from the bending portion 7.

In addition, in this state, swaging is performed in which the first portion A of the wire locking pin 22 is pressed in a direction of an arrow S3 in FIG. 11. Due to the swaging, a width W2 shown in FIG. 11 of the first portion A of the wire locking pin 22 deforms and the first portion A becomes larger than a width (refer to FIG. 12) of the long side L2 of the lateral hole 20ap as indicated by a width W2A shown in FIG. 12. Accordingly, the wire locking pin 22 is fastened to the lateral hole 20ap of the first bending piece 20a.

As described above, according to the embodiment, in a fixing structure of a specific bending piece (the first bending piece 20a) among the plurality of bending pieces 20 of the bending portion 7 and one end (distal end) of the bending wire 21, a simple structure of mounting the wire locking pin 22 that locks the one end of the bending wire 21 to the first bending piece 20a by pressing and fastening (swaging) is adopted. Accordingly, secure fixing can be performed by a simple operation (swaging) without having to perform complicated precision operations such as brazing that have conventionally been performed. Therefore, simplification of operations in the manufacturing process of the bending portion 7 can be realized and a contribution can be made toward reducing the number of assembly steps.

In addition, second swaging is performed in which the third portion C of the wire locking pin 22 is pressed in a direction from an inside of the first bending piece 20a to radially outward. Due to the second swaging, a width W3 of the third portion C or the wire locking pin 22 deforms and the third portion C becomes larger than a width of the long side L2 of the lateral hole 20ap.

In addition, since a configuration of each component (a bending piece or a wire locking pin) may be simple, a contribution can be made toward significantly reducing manufacturing cost. At the same time, a structure that readily enables mass production of the bending portion can be realized.

The structure of the bending portion of the endoscope according to the present embodiment can be applied to single-use endoscopes and, in particular, to endoscopes an object of examination, observation, and treatment of which is an inside of a gastrointestinal system. However, the structure according to the present disclosure is not limited to such an application. For example, by taking resistance qualities or the like of the fixing structure into consideration, the structure of the present disclosure can be similarly adopted with respect to so-called reusable endoscopes that can be repeatedly used by performing sterilization and disinfection after each use.

Another embodiment different from the above-described embodiment is shown in FIGS. 13 to 16. The other embodiment of the present disclosure is an embodiment in which a wire locking pin and a first bending piece are welded together by laser irradiation.

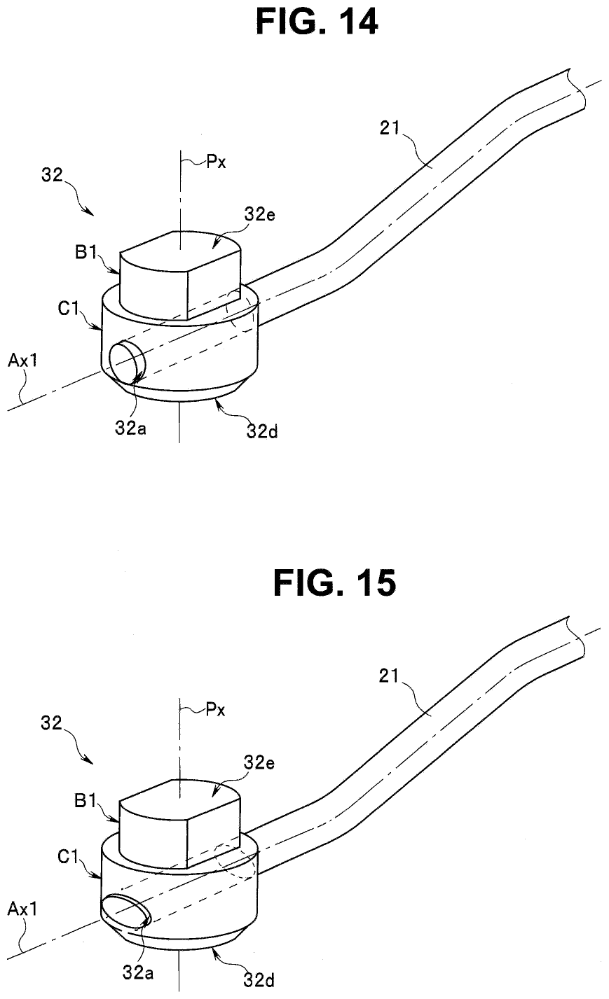

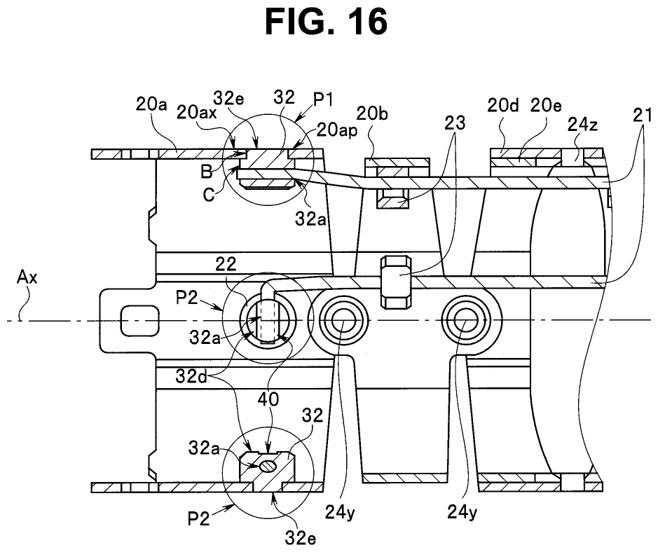

FIG. 13 is an external perspective view showing the wire locking pin in a state (single component) prior to being mounted to the bending piece in the other embodiment of the present disclosure. FIG. 14 is an external perspective view showing a state where one end of a bending wire is locked by the wire locking pin shown in FIG. 13. FIG. 15 is a view showing a situation where the through-hole is deformed by applying swaging to the wire locking pin shown in FIG. 14. FIG. 16 is an enlarged sectional view of a substantial part showing, in an enlarged manner, a section in a vicinity of a distal end side of a bending portion in the present embodiment.

The configuration of the present embodiment is basically the same as the configuration of the above-described embodiment. Therefore, in the following description, the same configuration portions as in the above-described embodiment are omitted in illustration and the drawings shown in the above-described embodiment are referred to. In FIGS. 13 to 16, which are illustrated as the configuration of the present embodiment, the same configuration portions as in the above-described embodiment are marked with the same reference signs, and the detailed descriptions of such configuration portions are omitted. Portions that differ from the above-described embodiment are described in detail below.

As shown in FIGS. 13 and 14, etc., the wire locking pin 32 of the present embodiment is a member that holds one end of the bending wire 21 and is fixed to the first bending piece 20a (refer to FIG. 4), to thereby fix the bending wire 21 to the first bending piece 20a. For this purpose, the first bending piece 20a is provided with a plurality of lateral holes 20ap in an approximately rectangular shape for arranging and fixing the wire locking pin 32.

The wire locking pin 32 has a second portion B1 and a third portion C1, as shown in FIG. 13. Here, the second portion B1 is a portion that is arranged at a position corresponding to the lateral hole 20ap of the first bending piece 20a in the state after assembly (refer to FIG. 16). The third portion C1 is a portion that is arranged so as to protrude toward an inner circumferential side of the first bending piece 20a in the same state shown in FIG. 16.

That is, in contrast to the wire locking pin 22 in the above-described embodiment, in the wire locking pin 32 of the present embodiment, the second portion B of the wire locking pin 22 corresponds to the second portion B1 of the wire locking pin 32, and the third portion C of the wire locking pin 22 corresponds to the third portion C1 of the wire locking pin 32. It also differs that the first portion A of the wire locking pin 22 is not provided to the wire locking pin 32.

The second portion B1 corresponds to a portion that is laser-welded to the bending piece 20a when the wire locking pin 32 is mounted in the lateral hole 20ap of the first bending piece 20a. Therefore, in a state before the wire locking pin 32 is mounted in the lateral hole 20ap of the first bending piece 20a, that is, a state where the wire locking pin 32 exists as a single component, the length of the second portion B1 in the long axis Px direction is formed to be approximately the same dimension as the wall thickness of the first bending piece 20a.

A through-hole 32a is formed in the third portion C1. The through-hole 32a is formed through the third portion C1 in a direction along an axis Ax1 orthogonal to the long axis Px of the wire locking pin 32. The through-hole 32a is a portion that holds the bending wire 21 when one end of the bending wire 21 is inserted as shown in FIG. 14.

Furthermore, the wire locking pin 32 is subjected to swaging in which the bending wire 21 is pressed in the direction of the long axis Px in a state where the bending wire 21 is held between inner wall surfaces of the through-hole 32a. This swaging deforms the through-hole 32a in the third portion C1 of the wire locking pin 32 as shown in FIG. 15, and the bending wire 21 is fastened to the wire locking pin 32. This swaging forms a concave portion 40 (refer to FIG. 16) on an end surface 32d of the third portion C1 (the surface facing the center direction of the bending piece 20a when the wire locking pin 32 is mounted to the bending piece 20a) approximately parallelly to the axis Ax1.

The wire locking pin 32 in which one end of the bending wire 21 is fastened in the through-hole 32a of the wire locking pin 32 is inserted through the lateral hole 20ap from the inner circumferential side of the first bending piece 20a (refer to FIG. 16). At this time, the second portion B1 of the wire locking pin 32 is first inserted through the lateral hole 20ap of the first bending piece 20a. Then, a flange surface 32b (refer to FIG. 13) of the third portion C1 abuts against the inner circumferential surface of the first bending piece 20a. This locks the insertion of the wire locking pin 32. At this time, the third portion C1 is arranged so as to protrude toward the inner circumferential side of the first bending piece 20a. An end surface 32e of the second portion B1 (the surface facing the outer direction of the bending piece 20a when the wire locking pin 32 is mounted to the bending piece 20a) is arranged approximately flush with an outer circumferential surface 20ax (refer to FIG. 16) of the first bending piece 20a.

In this state, the end surface 32e of the second portion B1 and the periphery of the lateral hole 20ap are laser irradiated by a laser irradiator, to thereby weld together the wire locking pin 32 and the first bending piece 20a. After the laser welding, the convexity created by the laser may be polished.

Thus, in the case of fixing the wire locking pin 32 to the first bending piece 20a by laser welding, it is enabled to eliminate any portion protruding on the outer surface 20ax of the first bending piece 20a, to thereby reduce the outer diameter of the bending portion.

The present disclosure is not limited to the embodiment described above and it goes without saying that various modifications, applications, and the like are possible without departing from the gist of the present disclosure. Furthermore, the above embodiment includes disclosures at various stages, and various disclosures can be extracted by appropriately combining the plurality of disclosed constituent features. For example, several constituent features can be deleted from all of the constituent features presented in the embodiment described above. A configuration from which the constituent features have been deleted can be extracted as a disclosure. Furthermore, constituent elements over different embodiments may be appropriately combined. The present disclosure is not limited by specific embodiments.

What is claimed is:

1. A bending portion for use with an endoscope, comprising:
a plurality of bending pieces, wherein adjacent bending pieces of the plurality of bending pieces are rotatably coupled to each other by at least one coupling fastener;
a wire configured to transmit a traction operating force to a first bending piece included in the plurality of bending pieces; and
a pin located in a first hole in the first bending piece,
wherein a first end portion of the pin protrudes from the first hole past an outer circumference surface of the first bending piece,
wherein a second end portion of the pin protrudes from the first hole past an inner circumference surface of the first bending piece,
wherein a third portion of the pin is between the first end portion and the second end portion, and the third portion of the pin contacts a surface of the first hole,
wherein the first end portion has a first width dimension that is larger than an opening dimension of the first hole, and
wherein the wire is attached to the second end portion.

2. The bending portion according to claim 1, wherein the second end portion has a second width dimension that is larger than the opening dimension of the first hole.

3. The bending portion according to claim 1, wherein the second end portion includes a through-hole in which the wire is located.

4. The bending portion according to claim 3, wherein the pin extends in a longitudinal direction from a first end surface of the first end portion to a second end surface of the second end portion, and
wherein an axis of the through-hole intersects the longitudinal direction of the pin.

5. The bending portion according to claim 1, wherein the bending portion is configured to be attached to an insertion portion of the endoscope,
wherein the plurality of bending pieces is arranged in a longitudinal direction of the bending portion to form a longitudinally arranged plurality of bending pieces, and the first bending piece is located at a distalmost end of the longitudinally arranged plurality of bending pieces, and
wherein the first bending piece includes a second hole that opposes the first hole across a longitudinal axis of the longitudinally arranged plurality of bending pieces.

6. The bending portion according to claim 5, wherein the first bending piece further includes a third hole and a fourth hole,
wherein the third hole opposes the fourth hole across the longitudinal axis, and
wherein a first straight line connects a center of the first hole and a center of the second hole, a second straight line connects a center of the third hole and a center of the fourth hole, and the first straight line intersects the second straight line.

7. The bending portion according to claim 1, wherein the plurality of bending pieces is integrally formed from a metal plate with a predetermined thickness.

8. The bending portion according to claim 1, wherein the wire is inserted through a plurality of guide portions respectively arranged at each of the plurality of bending pieces.

9. The bending portion according to claim 8, wherein the at least one coupling fastener includes a first coupling fastener and a second coupling fastener, and
wherein, in each of the plurality of bending pieces, the first coupling fastener and the second coupling fastener are located at positions separated in a circumferential direction from positions of the guide portions.

10. An endoscope, comprising:
the bending portion according to claim 1.

11. A manufacturing method of a bending portion of an endoscope, comprising:
coupling a first bending piece and a second bending piece to each other with a coupling fastener, the first bending piece including a first hole extending through a circumferential wall of the first bending piece,
fixing a first end portion of a pin to a first end of a wire, the pin having a length which exceeds a thickness of the circumferential wall of the first bending piece;
inserting the pin into the first hole from an inner side of the first bending piece to position a second end portion of the pin to protrude from the first hole past an outer circumference surface of the circumferential wall of the first bending piece; and
attaching the pin to the first bending piece.

12. The manufacturing method according to claim 11, wherein attaching the pin to the first bending piece includes deforming the second end portion of the pin so that a second width dimension is larger than an opening dimension of the first hole, the second width dimension being in a common orientation with the opening dimension.

13. The manufacturing method according to claim 11, wherein the second bending piece includes a wire guide portion, and
wherein the method further comprises:
passing the first end of the wire through the wire guide portion before fixing the first end portion of the pin to the first end of the wire.

14. The manufacturing method according to claim 11, wherein the second bending piece includes a wire guide portion, and
wherein the method further comprises:
positioning the wire in the wire guide portion after fixing the first end portion of the pin to the first end of the wire.

15. The bending portion according to claim 1, wherein the first width dimension is in a common orientation with the opening dimension.

16. The endoscope according to claim 10, wherein the first width dimension is in a common orientation with the opening dimension.

17. The bending portion according to claim 3, wherein the at least one coupling fastener includes a first coupling fastener coupling the first bending piece and a second bending piece proximally adjacent to the first bending piece,
wherein an axis of the through-hole intersects a longitudinal direction of the plurality of bending pieces, and
wherein the pin is disposed at a circumferential position around a center axis of the first bending piece corresponding to a circumferential position of the first coupling fastener.

18. The bending portion according to claim 3, wherein an axis of the through-hole is along a longitudinal direction of the plurality of bending pieces.

19. The bending portion according to claim 1, wherein the at least one coupling fastener includes a first coupling fastener and a second coupling fastener coupling the first bending piece and a second bending piece, the second bending piece located proximally adjacent to the first bending piece,
wherein the pin includes a first pin and a second pin, the first pin includes a first through-hole in which a first wire is located, the second pin includes a second through-hole in which a second wire is located,
wherein the second pin is located in a second hole in the first bending piece, the second pin includes:
a first end portion protruding from the second hole past the outer circumference surface of the first bending piece,
a second end portion protruding from the second hole past the inner circumference surface of the first bending piece,
a third portion between the first end portion of the second pin and the second end portion of the second pin, and the third portion of the second pin contacts a surface of the second hole,
wherein the first end portion of the second pin has a first width dimension that is larger than an opening dimension of the second hole,
wherein a first axis of the first through-hole and a second axis of the second through-hole intersect a longitudinal direction of the plurality of bending pieces,
wherein the first pin is disposed at a first circumferential position around a center axis of the first bending piece corresponding to a first circumferential position of the first coupling fastener, and
wherein the second pin is disposed at a second circumferential position around the center axis of the first bending piece corresponding to a second circumferential position of the first coupling fastener.

20. The bending portion according to claim 1, wherein the at least one coupling fastener includes a first coupling fastener and a second coupling fastener coupling the first bending piece and a second bending piece, the second bending piece located proximally adjacent to the first bending piece,
wherein the pin includes a first pin, a second pin and a third pin, the first pin includes a first through-hole in which a first wire is located, the second pin includes a second through-hole in which a second wire is located, the third pin includes a third through-hole in which a third wire is located,
wherein the second pin and the third pin are each located in a second hole and a third hole in the first bending piece, the second pin and the third pin each include:
a first end portion of the second pin protruding from the second hole past the outer circumference surface of the first bending piece, a first end portion of the third pin protruding from the third hole past the outer circumference surface of the first bending piece,
a second end portion of the second pin protruding from the second hole past the inner circumference surface of the first bending piece, a second end portion of the third pin protruding from the third hole past the inner circumference surface of the first bending piece,
a third portion of the second pin between the first end portion of the second pin and the second end portion of the second pin, and the third portion of the second pin contacts a surface of the second hole, a third portion of the third pin between the first end portion of the third pin and the second end portion of the third pin, and the third portion of the third pin contacts a surface of the third hole,
wherein each of the first end portion of the second pin and the first end portion of the third pin have a first width dimension that is larger than each of an opening dimension of the second hole and an opening dimension of the third hole,
wherein a first axis of the first through-hole and a second axis of the second through-hole intersect a longitudinal direction of the plurality of bending pieces,
wherein a third axis of the third through-hole is along the longitudinal direction of the plurality of bending pieces,
wherein the first pin is disposed at a first circumferential position around a center axis of the first bending piece corresponding to a first circumferential position of the first coupling fastener, and
wherein the second pin is disposed at a second circumferential position around the center axis of the first bending piece corresponding to a second circumferential position of the first coupling fastener.
